(19) United States
(12) Patent Application Publication
(10) Pub. No.: US 2008/0046069 A1
(43) Pub. Date: Feb. 21, 2008

(54) Flexible Delivery System

(76) Inventors: Duncan Keeble, Oxfordshire (GB); Anthony Jones, Oxfordshire (GB)

Correspondence Address: DEWITT ROSS & STEVENS S.C., 8000 EXCELSIOR DR, SUITE 401, MADISON, WI, 53717-1914, US

(21) Appl. No.: 10/595,371

(22) PCT Filed: Oct. 15, 2004
(86) PCT No.: PCT/GB04/04385

§ 371 Date: Feb. 16, 2007

(30) Foreign Application Priority Data
Oct. 15, 2003 (GB) 0324173.4

(51) Int. Cl. A61F 2/06

(52) U.S. Cl. 623/1.23

(57) ABSTRACT

An articulated device for advancing a medical implant along a catheter comprises a plurality of segments (1, 12) arranged one after the other in line, each segment being hingeably connected to a single adjacent segment if it is at the end of the line and otherwise to two adjacent segments, whereby a medical implant mounted at one end of the device can be advanced through a catheter by pushing on the other end of the device, the hinged connections allowing the device to follow a curved path through the catheter.

The present application relates to an articulated device for advancing a medical implant along a catheter. In particular, it relates to flexible tubular or catheter-based delivery systems for introducing implants into patients through a remote point of entry. It improves on existing delivery systems that are used to place stent grafts into arteries, most commonly using an entry point at the iliac or common femoral artery, and to deploy the stent graft within the aorta.

Current stent grafts designed for implantation into the aorta are typically radially compacted by a factor of 4 so that a 28 mm diameter graft will require a delivery system with a diameter of the order of 7 mm.

While this diameter of delivery system is sufficiently small to permit surgery through minor incisions, it is difficult to achieve the degree of flexibility that is required to pass through the vascular tree to the delivery site.

Many stent graft delivery systems, such as the Zenith™ from Cook Inc, the Talent™ from Medtronic Inc and the Anneurx™ also from Medtronic Inc involve two key components: an outer sheath and an inner ‘retainer’ rod. In use, the stent graft is compacted and inserted into the end of the sheath and the retainer rod is inserted from the far end of the sheath until the retainer rod contacts the stent graft. By various means, the sheath and its contents are introduced through the vascular tree until that part of the sheath containing the stent graft is located at the desired landing site for the stent graft. The sheath is then pulled slowly backwards, but the stent graft is retained in position by the retainer rod. As the sheath is pulled further back, the stent-graft begins to emerge from the open end of the sheath and deployment is complete when the sheath has been pulled back to the point where its end is level with the end of the retainer rod.

U.S. Pat. No. 6,589,227 (William Cook Europe APS) discloses an endovascular device for delivery of an expandable prosthesis to a body lumen. The device is formed from multiple-filament groups of individual wire coils.

U.S. Pat. No. 6,464,716 (Innercool Therapies, Inc.) discloses an endovascular heat transfer device formed from a plurality of elongated articulated segments which are connected by flexible joints formed from bellows or flexible tubes. The device is used to control body temperature, particularly that of the brain in the control of hypothermia.

Other implantation devices are disclosed in U.S. Pat. No. 5,954,729 (Schneider (USA) Inc.) and EP 0 518 838 (AMS Medinvent SA).

In practice, the retainer rod must be made of a material which is sufficiently flexible to allow the delivery system to follow the curves of the arterial tree. However, the forces involved in deploying stent grafts can be quite high and the retainer rod may be axially compressed as the stent graft is being deployed. Such compression is undesirable because it reduces the accuracy of deployment and can be the cause of radial expansion of the retainer rod. This radial expansion can lock the retainer rod in the sheath, preventing further deployment of the device.

A further requirement of the retainer rod is that it should be able to transmit twisting of the handle of the delivery system through to the stent graft. In a successful delivery system, the position of the device needs to be accurately controlled in rotation so that features of the stent graft can be made to align with anatomy. When the retainer rod is too soft or elastic, control of the device from the handle is imprecise, making it difficult, for instance, to ensure that paired legs of a bifurcated graft lie in a plane parallel to their target vessels.

Thus the requirement for flexibility suggests soft materials for the retainer rod, whereas the requirements of torsion control and incompressibility suggest employing a stiff material.

In accordance with the invention, one partial solution to these contradictory requirements is to employ a hard material for the retainer rod, but to cut it into short segments which are free to articulate against each other.

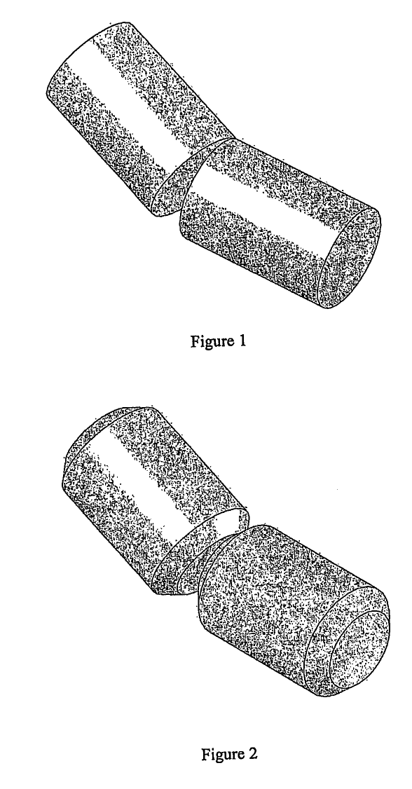

This solution is illustrated in FIG. 1 in which two segments of retainer rod are shown, articulated against each other to provide a flexible, incompressible retainer rod.

The solution relies upon the presence of the outer sheath to prevent the segments from migrating and is further compromised by the complete absence of a mechanism for transmitting torque from one segment to the next. It is obvious that a practical device will require a multiplicity of segments of the type illustrated in FIG. 1.

A further problem with this approach is that the composite retainer rod lengthens as it is flexed making the approach impractical for applications requiring high levels of flexibility.

An improvement over this first design is illustrated in FIG. 2 which employs segments of a hard material as before but in which abutting ends of the segments are chamfered so that the degree of articulation can be increased before the retainer rod lengthens.

Having established the principles illustrated in FIGS. 1 and 2 in which the retainer rod has a segmental construction and in which the abutting surfaces are modified to improve the characteristics of the ensemble it is possible to devise further modifications to the abutting surfaces to provide additional features.

In accordance with a first aspect of the invention, there is provided an articulated device for advancing a medical implant along a catheter, the device comprising a plurality of segments arranged one after the other in line, each segment being hingeably connected to a single adjacent segment if it is at the end of the line and otherwise to two adjacent segments, whereby a medical implant mounted at one end of the device can be advanced through a catheter by pushing on the other end of the device, the hinged connections allowing the device to follow a curved path through the catheter

The provision of segments which have hinged connections therebetween means that the segments can be formed from a relatively stiff material (such as a thermoset plastics material or even a metal or metal alloy) resulting in a device which is capable of transmitting a high torsional force from the operator to the medical implant.

In a preferred embodiment, the segments are formed from a glass-reinforced polyphenylene sulphide (provided under the trade mark Fortron® from Ticona). A device assembled from such segments is able to transmit a moment of 1 Newton metre and can sustain a compressive force of up to 760 Newtons with negligible shortening. In a preferred embodiment therefore the device is formed from a material which is able to transmit moment of at least 0.5 Nm, preferably at least 0.75 Nm, most preferable 1 Nm. The minimum sustainable compressive force with negligible shortening is preferably 300 N, more preferably 500 N and most preferably 750 N.

The segments are preferably detachable which allows for a device of any length to be assembled simply by increasing the number of segments.

In order to transfer torque effectively from one segment to another, at least one lateral process may be incorporated into the segment so that it will engage a corresponding elongated opening in the abutting segment.

Ideally the abutting surfaces have part spherical ends to allow the greatest degree of flexion between adjacent segments.

In a preferred embodiment each segment comprises a male part (which may comprise a ball and/or a pair of projections) and a female part (which may comprise a socket and/or a pair of slots) the male part of a segment being able to engage with the female part of an adjacent segment, and the female part being able to engage with the male part of an adjacent segment.

The connection between each segment is preferably of the “snap-fit” variety which allows for straightforward connecting of segments but prevents the segments from becoming detached too easily in use. For example, the mouth of the slots on the female part may be slightly narrower than the external diameter of the projections on the male part, so that a slight force needs to be applied to force apart the jaws of the slots and allow the projections to pass therethrough. The slot width then widens slightly beyond the slot mouth to accommodate the projections in a secure sliding fit. Although the segment as a whole is relatively stiff, it should in this embodiment be formed from a material which is able to resiliently flex to accommodate the projections on an adjacent segment.

Ideally, the practical design for a 21 French (7 mm diameter) delivery system employs segments which are 6 mm in diameter and 10.5 mm long. These dimensions can be scaled larger or smaller to cater for larger or smaller delivery systems. Thus a 10 French system will employ segments approximately 3 mm in diameter and a 50 French system will require segments 15 mm in diameter. The ratio of length to diameter of the segments is preferably 1.75:1 and workable designs can be made where this ratio is increased to 5:1 although manufacturing is less demanding and flexibility is improved if the ratio is limited to between 1.5:1 and 3:1. If some reduction in strength is acceptable, the ratio between the length and the diameter can be reduced to 1:1. It is inadvisable to make the segments shorter than their width because they are more inclined to ‘rock’ in the sheath and to cause jamming.

The maximum degree of articulation between any two adjacent segments is defined as the angle which the longitudinal axis of one segment makes with the longitudinal axis of the adjoining segment. This depends primarily on the nature of the hinge connection between the two segments. In the case of the ball and socket joint of the preferred embodiment, it depends on the relative sizes of the mouth of the socket and the external diameter of the segment at the part of the segment which is aligned with the mouth of the socket when the two segments are connected. In a preferred embodiment, the maximum degree of flexion is at least 15° and preferably up to 25°.

The segment is designed to be easily manufactured and injection moulding is a convenient technique to use, employing an appropriately hard and sterilisable plastic. Preferably, a single segment is designed so that features on a first abutting surface correspond with inverse features on the second abutting surface. It is possible to design segments which must be combined in pairs, although this is less convenient. With a single segment, multiple units can be stacked to form a long, rod-like structure, while each segment can be manufactured from a single injection moulding tool.

Preferably, each segment has a central axial hole that allows a guide wire and surrounding structures to pass therethrough. For example, the guide wire(s) may be retained in a tube having an outer wall which is smooth to reduce friction between the tube and the segments.

In one embodiment, the tube may be attached at either end to the end segments, so that the interior segments are retained therebetween. The advantage of this is that, if a segment does come detached from its adjacent segments, it is retained on the tube like a pearl on a necklace.

In some applications, there are advantages in assembling the device from groups of segments so that, for instance, the group of segments nearest the handle provide less flexibility than those at the tip. Thus the device may be comprised of at least two types of segments, the segments of the first type having different properties from the segments of the second type.

The walls of the segment are preferably slightly barrel-shaped so that even under extreme degrees of flexion the profile of the sheath over the segments is smooth and continuous.

An additional lumen may be provided in the form of a groove located off the central axis of the segment. It will be apparent to the skilled person that additional grooves can be placed at other points around the circumference of the segment.

A typical delivery system may require between 15 and 80 of the segments described, depending on the length of the device. However, in some embodiments, a greater number of segments may be employed (for example up to 1000) to attain an overall length of 2-3 metres.

At either end of the device modified segments, or end segments, can be used so that an effective interface is made between the segmental device and the handle components at one end, and the segmental device and the implant at the second end. In either case, the end segments will be designed to match the handle and implant components but it is desirable that those aspects of the end segments which interface with the segmental device retain all the mating features so that torque and lumens can be transmitted through to other components and to ensure that flexibility is retained.

In accordance with a second aspect of the invention, there is provided a kit comprising a device as defined above and a medical implant mounted on one end of the device.

In accordance with a third aspect of the invention, there is provided a segment for a device as defined above.

In accordance with a fourth aspect of the invention, there is provided a method of advancing a medical implant along a catheter comprising providing a device as defined above having an implant mounted on one end of the device, inserting said end of the device into the catheter, and pushing on the other end of the device.

A number of preferred embodiments of the invention will now be described with reference to the drawings, in which:

FIG. 1 illustrates a basic segmental retainer rod comprising simple, plane-faced cylindrical segments;

FIG. 2 illustrates an improved version of FIG. 1 in which the abutting faces have been chamfered to enable a greater degree of flexion to take place without a significant change in length occurring;

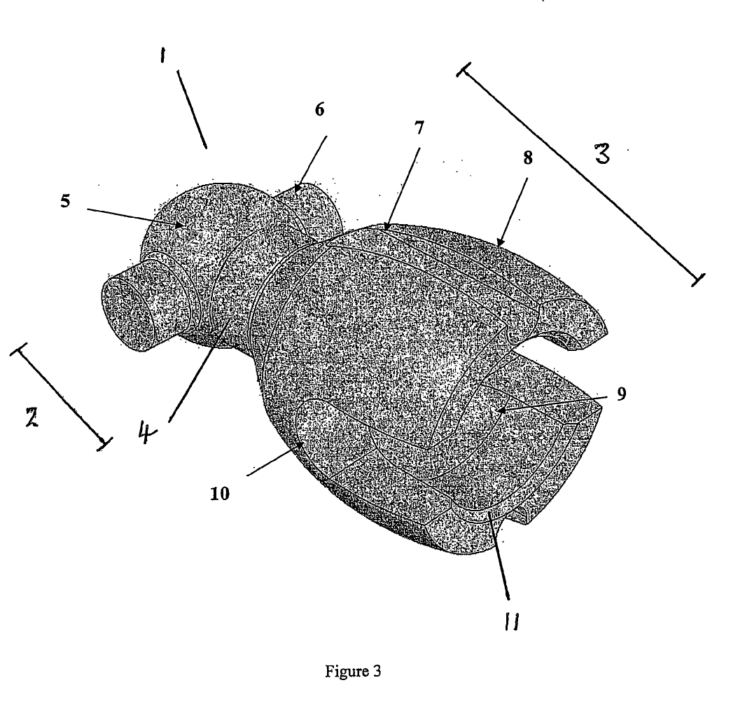

FIG. 3 illustrates a segment suitable for a device in accordance with the invention; and

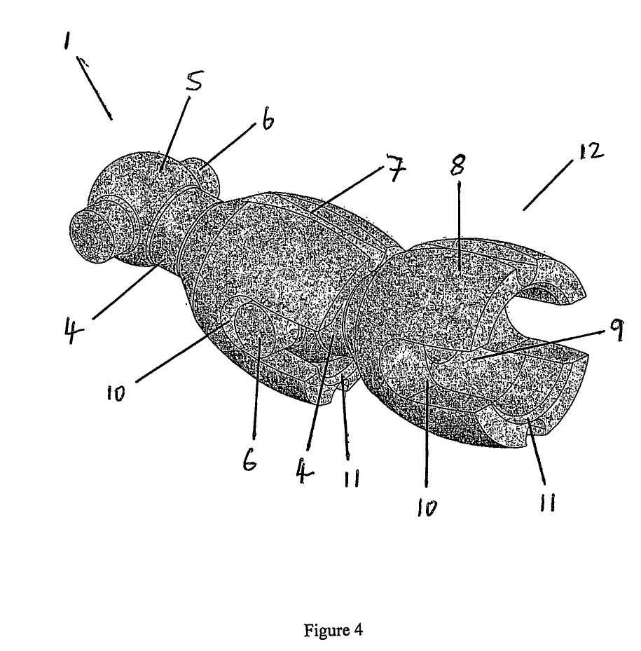

FIG. 4 illustrates two segments of the type shown in FIG. 3 connected as they would be in a device in accordance with the invention.

Turning first to FIG. 3, this illustrates a practical design of segment which employs the characteristics shown in FIGS. 1 and 2 but which includes additional features to transmit torque, to allow additional longitudinal structures and to maximise the smoothness of the outer sheath when flexed.

Segment 1 comprises an integral elongate element formed of a glass-reinforced polyphenylene sulphide with a length of approximately 10.5 mm and a width at its widest point of approximately 6 mm.

Segment 1 has male part 2 and female part 3 which meet at neck 4, and is configured so that male part 2 of one segment engages with female part 3 of an adjacent segment. A plurality of segments can therefore be linked end-to-end by connecting corresponding male and female parts.

In particular, male part 2 comprises ball 5 having pair of lugs 6 projecting laterally therefrom in an axis orthogonal to the longitudinal axis of segment 1. Female part 3 has socket joint 9 with lip 11, and a pair of slots 10 substantially parallel to the longitudinal axis of segment 1.

The mouth of each slot 10 is slightly narrower than the width of each lug 6, so that a slight force needs to be applied to the lug 6 to force the jaws of each slot 10 to flex slightly and move apart to allow lug 6 to pass therebetween. Each slot 10 then widens slightly beyond its mouth to accommodate each lug 6 in a sliding fit.

In use, lugs 6 of an adjacent segment can be slotted into slots 10 so as to seat ball 5 in socket 9 to form a ball and socket joint.

It will be appreciated that female part 3 is laterally wider than male part 2 which has to be sized so as to fit into female part 3. Accordingly, it is the outer surface of female part 3 that is more likely to come into contact with the inner wall of a catheter into which segment 1 has been inserted, and it is for this reason that outer walls 8 of female part 3 are curved so as to provide a smooth surface for rebutting the inner wall of the sheath even under extreme degrees of flexion.

A lumen (not shown) is provided along the longitudinal axis of the segment at or close to the centre, to allow a guide wire and surrounding structures to pass therethrough.

Two cut-out channels 7 are provided in the outer wall of female part 3 of segment 1 to allow for the use of other guide wires or similar structures.

Turning to FIG. 4, two identical segments 1 and 12 are shown in a connected state with lugs 6 of segment 12 fully inserted into slots 10 of segment 1 and ball 5 of segment 12 (not shown) seated in socket 9 of segment 1 (not shown).

It can be seen from FIG. 4 that the width of neck 4 is less than the internal diameter of the mouth of socket joint 9, and thus there is sufficient space for segment 12 to rotate about the axis of its lugs 6 thereby allowing a degree of articulation between segment 1 and segment 12. In FIG. 4, segments 1 and 12 are shown in their fully flexed state, with segment 12 being rotated by about 15 degrees so that the outer surface of neck 4 of segment 12 abuts lip 11 of segment 1.

In use, between 15 and 80 segments are linked as shown in FIG. 4, the number depending on the length of the catheter into which the device is to be inserted. A medical implant is mounted on the end segment, which may be modified so as to receive the implant. At the end of the device distal to the implant, a modified segment having handles is employed, the handles being used to apply force to the device both along its longitudinal axis (to advance the implant through the catheter) and to rotate the device about its longitudinal axis so as to apply torsional force to rotate the implant. Such torsional force can be applied along the length of the device because of the lack of play between lugs 6 and slots 10 of adjacent segments.

In order to implant a stent graft, for example, in vivo, the stent graft is mounted on the end of a device according to the invention and is then inserted into an outer sheath and advanced until the stent graft is at the end of the outer sheath distal from the operator. The outer sheath can then itself be advanced down a catheter, and a catheter inserted into the vascular tree. When the distal end of the outer sheath is at the required implant site, the sheath is pulled slowly backwards with the stent graft being held in place by the inventive device. The operator can easily rotate the stent graft so as to place it accurately by rotating the handle at the end of the device. Moreover, pressure applied to the sheath in order to deploy the stent graft does not cause compression of the device, in contrast to prior art devices. Thus the device according to the invention can be successfully employed to implant stent grafts in vivo.

1. An articulated device for advancing a medical implant along a catheter, the device comprising a plurality of segments arranged one after the other in line, each segment being hingeably connected to a single adjacent segment if it is at the end of the line and otherwise to two adjacent segments, whereby a medical implant mounted at one end of the device can be advanced through a catheter by pushing on the other end of the device, the hinged connections allowing the device to follow a curved path through the catheter, characterised in that each segment is detachable from its adjacent segment(s).

2. A device as claimed in claim 1, wherein each segment comprises a male part and a female part, the male part of a segment being able to engage with the female part of an adjacent segment, and the female part being able to engage with the male part of an adjacent segment.

3. A device as claimed in claim 2, wherein the male part comprises a pair of projections and the female part comprises a slot for accepting the projections.

4. A device as claimed in claim 2, wherein the male part comprises a ball and the female part comprises a socket.

5. A device as claimed in claim 1, wherein the segments are formed from a material which is sufficiently stiff to allow a moment of at least 1 Newton metre to be transmitted through the device.

6. A device as claimed in claim 1 which includes from 15 to 80 segments.

7. A device as claimed in claim 1, wherein each segment has a lumen passing through its body along its longitudinal axis, so that the plurality of lumen substantially align to allow a guide wire to pass therethrough when the device is in use.

8. A device as claimed in claim 1, wherein each segment has a channel in its outer wall so that the plurality of channels substantially align to allow a guide wire to pass therethrough when the device is in use.

9. A device as claimed in claim 1, wherein the ratio of the length to the widest diameter of each segment is in the range 1:1 to 1:5.

10. A device as claimed in claim 1, wherein the maximum degree of articulation between the longitudinal axis of one segment and the longitudinal axis of an adjacent segment is at least 15.

11. A device as claimed in claim 1 in combination with a medical implant mounted on one end of the device.

12. A device as claimed in claim 11 wherein the medical implant is a vascular graft.

13. The device of claim 11 further comprising a delivery catheter.

14. (canceled)

15. A method of advancing a medical implant along a catheter comprising providing a device as claimed in claim 1 having an implant mounted on one end of the device, inserting said end of the device into the catheter, and pushing on the other end of the device.

16. An articulated device for advancing a medical implant along a catheter, the device comprising:
a. a catheter having a catheter interior passage;
b. multiple segments adjacently arrayed in a line within the catheter interior passage, wherein:
(1) each segment pivotally abuts any adjacent segments, whereby the line of segments may adopt a curved path within the catheter, and
(2) the segments are translatable within the passage, whereby the segment at one end of the line can:
(i) have a medical implant situated thereon, and
(ii) be advanced through at least a major portion of the length of the catheter interior passage to eject the medical implant from a passage exit.

17. The articulated device of claim 16 further comprising a passage defined within each segment, wherein the passages are aligned when the segments are arrayed in a line to define a passage extending axially along the arrayed segments.

18. The articulated device of claim 17 wherein the passage in each segment is situated on the outer circumference of each segment.

19. The articulated device of claim 17 wherein the passage in each segment extends through each segment spaced from the segment's outer circumference.

20. The articulated device of claim 16 further comprising a tube extending through the segments.

21. The articulated device of claim 19 wherein the tube is affixed to at least two segments which are spaced by intermediate segments.

22. The articulated device of claim 16 wherein each segment is resiliently snap-fit to at least one adjacent segment.

23. The articulated device of claim 16 wherein each segment bears one or more projections, each projection being engaged to an adjacent segment.

24. The articulated device of claim 22 wherein each segment bears a ball thereon, and wherein the projections extend from the ball.

25. The articulated device of claim 16 wherein segments have lengths, as measured along the line, which are less than or equal to their diameters.

26. The articulated device of claim 16 wherein segments have diameters of approximately 10 mm or less.
